(19) United States
(12) Patent Application Publication
(10) Pub. No.: US 2020/0390644 A1
(43) Pub. Date: Dec. 17, 2020

(54) PROGRAMMABLE THERAPEUTIC DEVICE, METHOD, AND SYSTEM

(71) Applicant: Addaday, Inc., Los Angeles, CA (US)

(72) Inventor: Victor Yang, Palos Verdes Estates, CA (US)

(21) Appl. No.: 16/880,064

(22) Filed: May 21, 2020

Related U.S. Application Data
(60) Provisional application No. 62/860,191, filed on Jun. 11, 2019.

(51) Int. Cl. A61H 23/00; A61H 9/00; A61H 23/02; G16H 40/67; G16H 20/30

(52) U.S. Cl. CPC A61H 23/006; A61H 9/0007; A61H 23/008; A61H 23/02; A61H 2201/5097; G16H 20/30; A61H 2201/5043; A61H 2201/5035; A61H 2201/5038; G16H 40/67

(57) ABSTRACT

A programmable therapeutic device for use with individualized massage therapy. The therapeutic device is capable of receiving and executing custom-made, individually tailored massage therapy scripts, for use with aiding users with effective pain and stress relief. The programmable therapeutic device may provide one or more of vibration, pulse, percussion, oscillation, electrical stimulation, and combinations thereof.

### CROSS-REFERENCE TO RELATED APPLICATIONS

This Application claims the benefit of U.S. Provisional Patent Application No. 62/860,191, filed on Jun. 11, 2019, titled “PROGRAMMABLE THERAPEUTIC DEVICE, METHOD, AND SYSTEM”, the contents of which is expressly incorporated herein by this reference as though set forth in their entirety.

### FIELD OF USE

The present disclosure relates generally to the field of massage devices, and more specifically to a programmable massage system capable of changing its method of therapy to meet the individual health and medical needs of the user.

### BACKGROUND

Massages and related types of physical therapy are regularly used by many individuals, whether to provide relief from muscle aches and pains or to simply aid in relaxation and stress relief. In particular, motorized massage therapy devices, such as massage wands and massage chairs, have become increasingly popular for such purposes.

However, currently existing massage therapy devices only allow for extremely limited customization and tend to operate under a “one size fits all” approach. Users of such existing massage therapy devices are unable to program the device to provide a uniquely tailored, individual massage therapy experience. Current devices are limited either to a small number of preprogrammed patterns, or only allow users to vary the speed settings or the position of the massage device manually in real time. Traditional systems are limited because they cannot execute new massage patterns that are optimized specifically for a patient's symptoms. Users cannot reprogram the massager to execute new and different algorithms, programs, or scripts. Nor can the users author their own unique algorithms to suit their individual massage and therapeutic needs.

Thus, there is a need for a programmable therapeutic device, method, and system, which would allow users to have the flexibility to create, share, and use unique therapy scripts based on their individual medical needs.

### SUMMARY

To minimize the limitations in the cited references, and to minimize other limitations that will become apparent upon reading and understanding the present specification, the programmable therapeutic device, method, and system disclosed herein discloses a programmable therapeutic device and method of using this programmable therapeutic device to create, program, and execute individually-tailored massage scripts.

One embodiment may be a programmable therapeutic device capable of executing massage therapy scripts and other therapeutic scripts.

One embodiment may be a programmable therapeutic device capable of executing massage therapy scripts, wherein the programmable therapeutic device is capable of varying the pulse, vibration, percussive strength, and electrical stimulation provided by the programmable therapeutic device. The device itself may be a handheld portable massage device, a massage chair, or a device that is designed to engage with various body parts, such as hands, feet, arms, legs, head, or torso

One embodiment may be a programmable therapeutic device capable of executing massage therapy scripts, wherein the therapy scripts are authored by a doctor, physical therapist, chiropractor, specialist, or other medical professional based on symptoms experienced by the patient.

One embodiment may be a programmable massage therapy apparatus that uses a software application to communicate massage therapy scripts directly to the patients' programmable therapeutic device based on symptoms experienced by the patient. The massage therapy scripts may include instructions for the programmable therapeutic device to execute one or more massage therapy techniques, such as such as pulses, vibrations, percussions, oscillations, and/or transcutaneous electrical nerve stimulation (TENS). The software application may be executed on a computer, smart phone, tablet, laptop, and/or a device dedicated for creating scripts through a template program.

Another embodiment may be a method of authoring individually tailored massage therapy scripts, and subsequently delivering these therapy scripts to a programmable therapeutic device for execution. The delivery may be one-time uploads or provided on a subscription basis.

One embodiment may be a programmable massage therapy apparatus comprising a programmable therapeutic device and a counterpart programmable therapy template device. The template device may be configured to allow users and professionals to author therapy scripts by mimicking actions illustrated by a medical profession on a screen by gesture. The template device may be a dedicated computerized device with a display and input controls, or it may be a software application running on a computer, smartphone, or tablet that has a display and input controls (touch screen, mouse, keyboard, and the like). The graphical user interface of the template device not only allows professionals to intuitively author scripts, but also allows them to select to which patients to send the newly created therapy scripts.

In another embodiment, the created therapy scripts may be transmitted to the programable therapy device wirelessly. In other embodiments, the scripts may be uploaded through a wired connection or through a USB flash drive or memory card.

In one embodiment a medical practitioner may send a patient home with a programmable therapeutic device for continued therapy after an initial consultation. In another embodiment, the medical practitioner may author and transmit a therapy script to a user's programmable therapeutic device without the need to ever meet the user in person. In this situation, the medical practitioner may use information from the user regarding the user's health in order to create a therapy script that is tailored to the specific user's needs.

One embodiment may be a programmable therapeutic device capable of receiving software instructions for specific therapeutic massage scripts. The software instructions contained in the massage therapy scripts may manipulate various aspects of the programmable therapeutic device, such as motor vibration (duration, speed, and intensity), massage percussion (duration, speed, and intensity), and electrical stimulation (pulse intensity and duration). The software instructions may be delivered wirelessly or through installation of hardware, such as a flash drive, USB, or memory card.

One embodiment may be a software application for authoring massage therapy scripts, allowing a user to customize the massage therapy routine(s) executed on the user's programmable therapeutic device. The software application may allow users to vary the motor vibration, massage percussion, and electrical stimulation of the therapy script based on touch settings performed on a touch screen. The settings may comprise intensity and/or duration, and may be textured movements with haptic feedback.

In some embodiments, the software application of the present disclosure may allow for members of the medical massage therapy community to author therapy scripts to be shared online. Members outside the medical massage therapy community may also author therapy scripts for online sharing and downloading. In one embodiment, community-authored therapy scripts may be distributed online via social media networks and/or software delivery platforms (App Stores).

In another embodiment, a user of a programmable therapeutic device may browse an online catalog of massage therapy scripts and download the desired script(s) for execution by the user's individual programmable therapeutic device. In one embodiment, users may download therapy scripts from online catalogs for a fee. In another embodiment, therapy scripts may be downloaded for no monetary charge. In some embodiments, the online catalog of downloadable massage therapy scripts may function similarly to a conventional music purchasing website.

One embodiment may be a programmable therapeutic device that includes bio-feedback sensors, which provide information to the user based on the duration and ultrasound.

The device and system of the present disclosure may allow the user to use techniques commonly administered by a physical therapist, chiropractor, Doctor of Physical Therapy (“DPT”) or sports massage therapist. These techniques may include, but are not limited to, pulses, vibrations, percussions, oscillations, and/or electrical stimulation.

The devices and systems of the present disclosure may improve the therapeutic experience for every individual. Every experience can become unique and uniquely authored. The same device can create a massage that is soft and tender or firm and strong—it just depends on what massage therapy script was executed on the device.

Other features and advantages inherent in the disclosed programmable therapeutic device and methods for programming scripts for a programmable therapeutic device, besides those which are claimed and disclosed, will become apparent to those skilled in the art from the following detailed description and its accompanying drawings.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps, which are illustrated. When the same numeral appears in different drawings, it refers to the same or like components or steps.

FIG. 1 is an illustration of a programmable therapeutic device.

FIG. 2 is an illustration of one embodiment of a programmable percussion therapeutic device.

FIG. 3 is an illustration of another embodiment of a programmable therapeutic device that provides vibration and percussion therapy.

FIG. 4 is an illustration of a graphic display of the programmable therapeutic device shown in FIG. 3.

FIG. 5 is an illustration of one embodiment of a programable therapeutic device with a display.

FIG. 6 is an illustration of an interior cut-away view one embodiment of a programable therapeutic device with a display.

FIG. 7 is an illustration of one embodiment of a therapy script authoring template.

FIG. 8 is an illustration of one embodiment of a system for programming a therapy device.

### DETAILED DESCRIPTION OF THE ILLUSTRATIVE EMBODIMENTS

In the following detailed description, numerous specific details are set forth in order to provide a thorough understanding of various aspects of one or more embodiments. However, these embodiments may be practiced without some or all of these specific details. In other instances, well-known methods, procedures, and/or components have not been described in detail so as not to unnecessarily obscure aspects of embodiments.

While multiple embodiments are disclosed, still other embodiments will become apparent to those skilled in the art from the following detailed description. As will be realized, these embodiments are capable of modifications in various obvious aspects, all without departing from the spirit and scope of protection. Accordingly, figures and the detailed descriptions thereof are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about,” may refer to a deviance of between 0.0001-10% from the indicated number or range of numbers.

The present specification discloses systems and methods for authoring an individually tailored massage therapy script and delivering said massage therapy script to a programmable therapeutic device which executes the massage therapy script.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

As will be appreciated by one skilled in the art, the systems and methods may take the form of an entirely hardware embodiment, an entirely software embodiment, or an embodiment combining software and hardware embodiments. Furthermore, the systems and methods may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, compact discs, read-only-memory (CD-ROMs), optical storage devices, or magnetic storage devices.

Embodiments of the systems and methods are described below with reference to schematic diagrams, block diagrams, and flowchart illustrations of methods, systems, apparatuses and computer program products. It will be understood that each block of the block diagrams, schematic diagrams, and flowchart illustrations, and combinations of blocks in the block diagrams, schematic diagrams, and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

In the following description, certain terminology is used to describe certain features of one or more embodiments. For purposes of the specification, unless otherwise specified, the term “programmable therapeutic device” generally refers to a device that provides physical therapy or relief, including, but not limited to, aa portable massager, a handheld massaging device, a massager chair, an electrical stimulation device, an oscillation massage device, a percussion massager, a vibration massager, a pulse massager, a limb massaging device, and/or any combination thereof.

As used herein, the terms “script”, “therapy script”, or “therapeutic script”, generally refer to a series of programmable instructions which are interpreted and executed by the programmable therapeutic device.

As used herein, the terms “deliver” and “delivered” refer to any means for transmitting a script to a programmable therapeutic device, such as by direct installation via a USB flash drive or other portable media (such as CDs, DVDs, etc.), or by wireless download.

As used herein, the term massage “therapy technique”, “physical therapy”, and “massage therapy techniques”, includes, but is not limited to, pulses, vibrations, percussions, oscillations, and electrical stimulations.

One embodiment may be a programmable therapeutic device capable of executing massage therapy scripts.

Another embodiment may be a programmable massage therapy apparatus comprising a programmable therapeutic device and a counterpart programmable therapy template device. The template device, preferably used by a massage therapy professional, may further comprise a graphical user interface (GUI) which allows for easy programming and authoring of massage therapy scripts. Once a therapy script has been authored, it may be delivered to the user and/or the device wirelessly, online (via Internet download), or by physical hardware installation (such as a memory card, CD, or USB flash drive).

One embodiment may be a programmable therapeutic device capable of executing massage therapy scripts, wherein the programmable therapeutic device is capable of providing and varying the pulse, vibration, percussive strength, and electrical stimulation of the therapeutic device.

Another embodiment may be a method of authoring individually tailored massage therapy scripts.

One embodiment may be a method of programming a programmable therapeutic device, comprising the steps:
providing a programmable therapeutic device;
wherein the programmable therapeutic device may be portable, handheld, and comprises a massage head, and/or may be a fixed device, such as a massage chair or boot;
providing a massage therapy script authoring user interface;
wherein the user interface allows for creation of scripts which vary any combination of the following: massage head pulse rate, massage head vibration rate, massage head percussive strength, and massage head TENS/electrical stimulation (TENS =transcutaneous electrical nerve stimulation);
delivering the massage therapy script to the programmable therapeutic device;
wherein the delivering may take the form of wireless transmission and download by the programmable therapeutic device, or direct hardware installation such as by Cat 5/6 connection, USB flash drive, memory chip, memory card, memory stick, CD, or DVD.

One embodiment may be a programmable therapeutic device:
wherein the programmable therapeutic device is capable of receiving delivered massage therapy scripts via wireless download or direct hardware installation such as by memory card, USB flash drive, CD, or DVD.

One embodiment may be a programmable massage therapy apparatus, comprising:
a programmable therapeutic device; and
a programmable therapy template device;
wherein the template device further comprises an interface (preferably a GUI touch screen) that enables the authoring of massage therapy scripts;
wherein the programmable therapeutic device is capable of receiving and executing massage therapy scripts, whether by wireless download or direct hardware installation; and
wherein a user of the programmable therapeutic device may select from a catalog of massage therapy scripts, to be executed on the device.

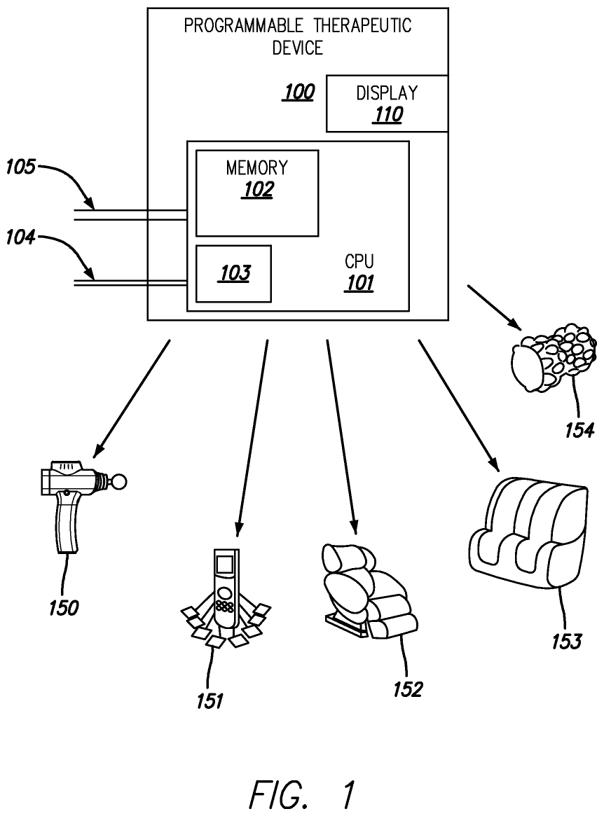

FIG. 1 is an illustration of a programmable therapeutic device. As shown in FIG. 1, the programmable therapeutic device 100 may be, but is not limited to, a portable handheld massage device 150, a TENS device 151, a massage chair 152, leg, foot, or individual body part massage device 153, oscillating roller massager 154. Preferably the device 100 may comprise a central processing unit (CPU) 101, which may comprise or be connected to a display 110, memory 102, a wireless transceiver 103, and input ports 104, 105. The device 100 receives therapeutic scripts or programs via the wireless transceiver 103, internet port 104, and/or input port 105, which may be a USB port, CD receiver, and/or a memory card receiver. The CPU 101 of the device 100 allows the device 100 to run the therapeutic scripts that are received so that the user can get massage or therapeutic relief that is individualized for that user or that is specifically requested/selected by that user. The device 100 may hold and run one script at a time, or the memory 102 and display 110 may allow the device 100 to hold numerous scripts, which may be selected for being run. Preferably, the user may always be able to add additional scripts and/or remove previously saved scripts. Thus, rather than being confined to those several programs that might come with a traditional therapeutic device, an unlimited number of new programs may be created and/or run on the device 100.

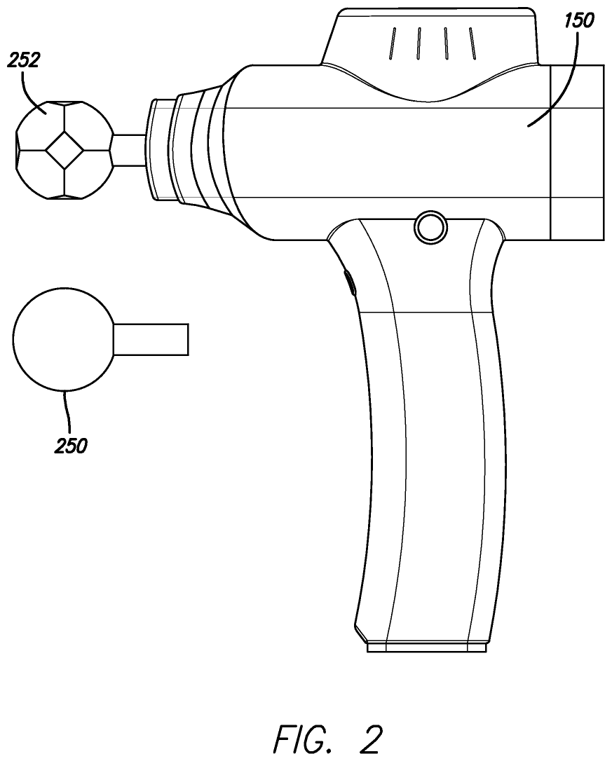

FIG. 2 is an illustration of one embodiment of a programmable percussion therapeutic device. As shown in FIG. 2, one embodiment of the device of the present disclosure may be a handheld massage device 150. Preferably the device 150 is a vibration, pulse, and/or percussion massage device that may have various massaging heads 252, 250. Preferably the massage head 252 is shorter than head 250. Shorter heads may preferably generate more vibration, longer rod generates more percussion.

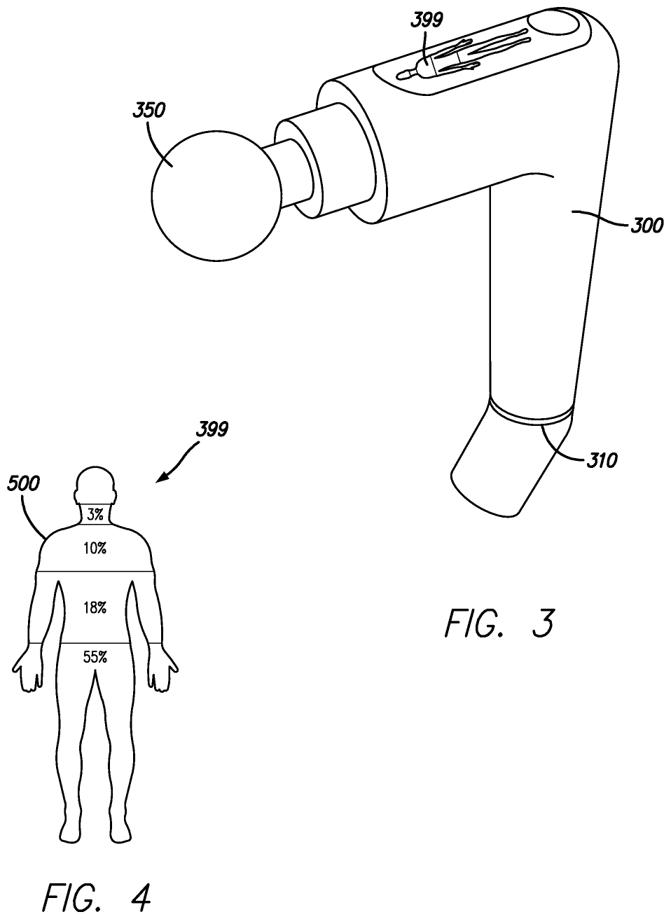

FIG. 3 is an illustration of another embodiment of a programmable therapeutic device that provides vibration and percussion therapy. The programmable therapeutic massage device 300 may have interchangeable heads 350, a display 399, and notification light 310. The notification light may inform the user as to the status of the device, such as when it has received a new program, when it is fully charged, when it is battery low, and the like. The display 399 may be an analog or digital display, such as a light emitting diode display. The display may assist the user in letting them know when, where, and for how long to massage various parts of the body.

FIG. 4 is an illustration of a graphic display of the programmable therapeutic device shown in FIG. 3. FIG. 4 shows that the display 399 of device 300 may be a depiction of a human body 500. The display 399 may inform the user how much time to spend on what body part, when to switch to a new body part, or generally assist the user in targeting those parts of the body that need to receive the massage therapy. In one embodiment, the human body depiction 500 may divide the body into zones or regions, including, but not limited to, hands, arms, legs, torso, shoulders, back, chest, feet, and neck.

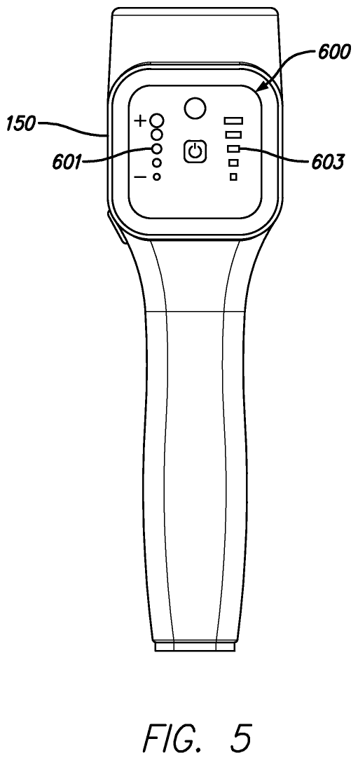

FIG. 5 is an illustration of one embodiment of a programable therapeutic device with a display. As shown in FIG. 5, the device therapeutic massage device 150 may have a display 600, analog or digital, which may allow the user to see what program is running, what the current strength 601 is, and what the current speed 603 is.

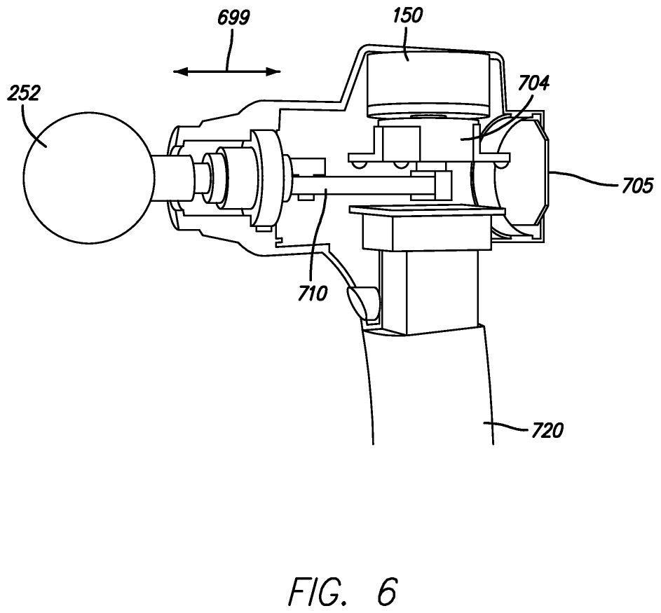

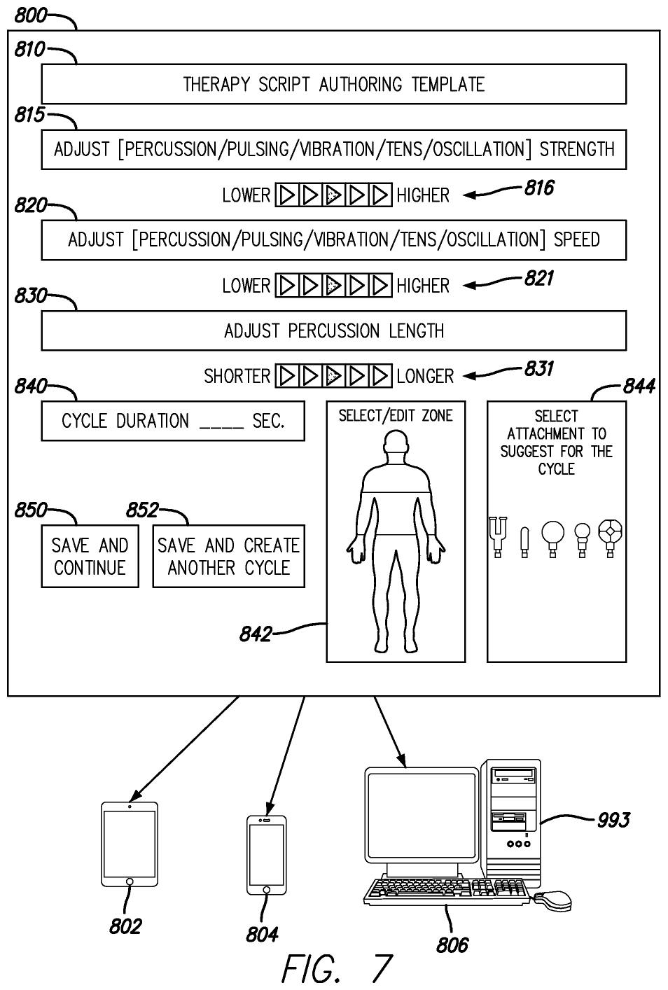

FIG. 6 is an illustration of an interior cut-away view one embodiment of a programable therapeutic device with a display. As shown in FIG. 6, the device 150 may be a therapeutic percussion device that has a motor 704 that moves the head 252 back and forth 699 via arm 710 to provide percussion therapy to the user. The motor FIG. 6 shows that the device 150 may have a swappable battery pack handle 720. The device 150 may have an integrated circuit board 705, which may comprise or be connected to the CPU, memory, transceiver, and/or input ports, which allow the device 150 to receive therapy scripts.

In one embodiment, a user may, through an appropriate GUI (graphical user interface), create a massage therapy script which adjusts various features of the massage therapy program over time. For example, a user may adjust the percussive strength or number of oscillations per second, as well as set a cycle of instructions which repeats after a determined interval. After the user has finished programming their desired massage therapy script, the script may then be exported to a programmable therapeutic device for execution. In this manner, the programmed script results in an individualized massage (or TENS) therapy experience for the end user.

In one embodiment, the device of the present disclosure may have an operating system capable of receiving massage/heating algorithms authored from a software application that either resides on a server and/or resident on a device. Memory on the device capable of storing the algorithms received by wireless, wired, or memory card. The device may have a motor capable of adjusting the length of Percussion and Vibration patterns. The rod adjustment sends different percussion and vibration notes to the body. Shorter rods generate more vibration, longer rods generate more percussion. The purpose of this device/system is to increase the amplitude/pattern so that the muscles are stimulated even closer to what at chiropractor or physical therapist would administer on an actual patient with soft tissue work by the hands. In many cases, because of the adjustable head and programmable massage algorithms, the system of the present disclosure may go above and beyond what human hands do to further improve the manipulation of soft tissue so that muscle recovery can accelerate.

FIG. 7 is an illustration of one embodiment of a therapy script authoring template. The therapy script template 800 may be displayed on the display screen of a template device, such as a tablet 802, smartphone 804, or personal computer 806. The template 800 may have a title 810, which is used by the user to quickly, accurately, and easily, create therapy programs or scripts to be run on programmable therapy devices. As shown in FIG. 7, the template 800 may be a graphical user interface that allows the user to adjust the strength 815 via scale 816. The user may adjust the speed 820 via scale 821. The user may adjust the length 830 via scale 816. Although scales are shown, any interface that lets the user raise and lower the desired therapy technique may be used.

FIG. 7 shows that the template may be used with specific devices, such as percussion, pulsing, vibration, TENS, and/or Oscillation. Once the desired strength, speed, and length (if necessary) are selected, the duration is selected 840, the zone is selected 842 (if needed), and the suggested attachment may be selected 844. Once the current cycle is ready, the user may save and continue 850 or may save and add another cycle to begin after the current cycle's duration expires. In this manner, the user quickly builds a complex therapeutic script to be “played” on a therapeutic device. The template may be a program with a graphical user interface that allows the user to create new therapy scripts and edit previously created scripts.

In one embodiment of the software application of the present disclosure, a user/creator/provider may create a massage script, which allows the control of any combination of the following as a function of time: massage head pulse rate, vibration speed, percussive strength, and TENS/electrical stimulation. After each of the scripts are authored by a user/creator, they can then be communicated to a programmable therapeutic device wireles sly or may be downloaded and saved into physical media, such as a memory card.

The massage therapy scripts may be subsequently loaded onto and executed (or ran) by a variety of programmable therapeutic devices. In one embodiment, the programmable therapeutic device may be a back, neck, or foot massager unit. In another embodiment, the programmable therapeutic device may be an oscillating foam roller. In another embodiment, the programmable therapeutic device may be a wireless TENS unit. In yet another embodiment, the programmable therapeutic device may be a massage gun or other handheld massage wand. In a preferred embodiment, the therapeutic device may offer one or more of percussing, pulsing, vibration, oscillation, electrical stimulation, and/or combinations thereof.

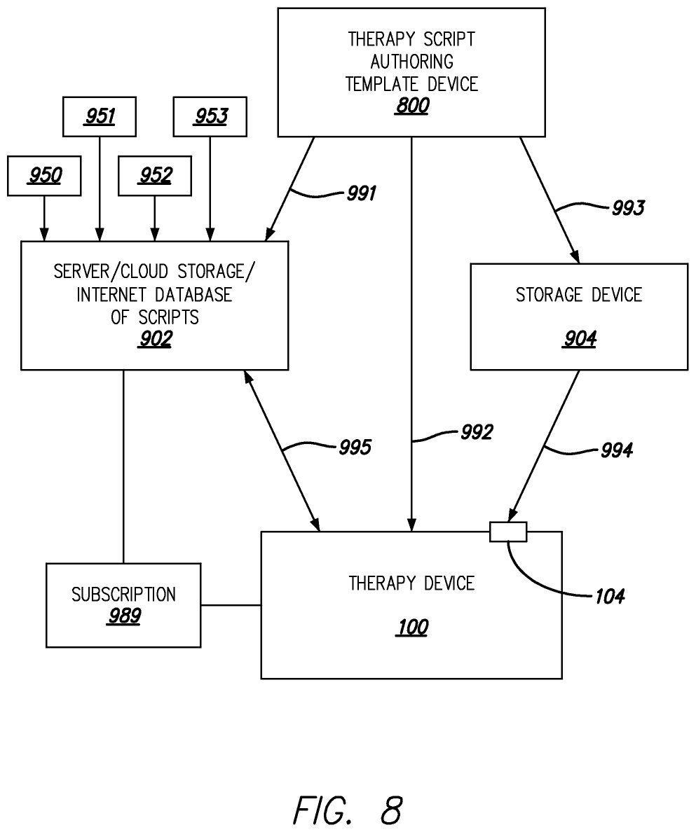

FIG. 8 is an illustration of one embodiment of a system for programming a therapy device. As shown in FIG. 8, the system may comprise therapy script authoring template devices 800 and therapy device 100. The therapy script authoring template devices 800, as detailed above in FIG. 7, may allow the user to create one or more therapy scripts that are configured to be run on a therapy device 100. Therapy scripts may be transmitted to the therapy device 100 from the therapy script authoring template device 800 via 993, 994 storage device 904, which may be connected to input 104. The therapy scripts may also be transmitted directly and wirelessly 992 to the therapy device 100 from the device 800. Alternatively, the therapy scripts may be sent 991 to a server/cloud storage/internet database 902. Then, the therapy device 100 may receive 995, 989 from the server/cloud storage/internet database 902 one or more of the created therapy scripts. In one embodiment, the user of the therapy device 100 may subscribe 989 in order to download the scripts uploaded by other therapy script authoring template devices 950, 951, 952, 953. In this manner, the user can share his/her authored scripts and benefit from scripts authored by others.

The subscription 989 service and/or therapy script authoring template software may also integrate the collection of personal health information from the user, including via personal devices, such as smart or fitness watches, fitness apps and fitness programs, or just collecting the data directly from the user. This collection of personal health information, along with, potentially, an actual or virtual physical examination of the user, may be used by the user or a medical/physical/massage therapist to create individually tailored therapy scripts.

Unless otherwise stated, all measurements, values, ratings, positions, magnitudes, sizes, locations, and other specifications, which set forth in this specification, including in the claims that follow, are approximate, not exact. They are intended to have a reasonable range, which is consistent with the functions to which they relate and with what is customary in the art to which they pertain.

In addition, The various illustrative logical blocks, modules, and circuits described in connection with certain embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, system-on-a-chip, or state machine. A processor may also be implemented as a combination of computing devices, e.g., a combination of a DSP and a microprocessor, a plurality of microprocessors, one or more microprocessors in conjunction with a DSP core, or any other such configuration.

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize many modifications may be made to this configuration without departing from the scope of the disclosed embodiments.

The foregoing description of the preferred embodiment has been presented for the purposes of illustration and description. While multiple embodiments are disclosed, still other embodiments will become apparent to those skilled in the art from the above detailed description, which shows and describes the illustrative embodiments. As will be realized, these embodiments are capable of modifications in various obvious aspects, all without departing from the spirit and scope of the present disclosure. Accordingly, the detailed description is to be regarded as illustrative in nature and not restrictive. Also, although not explicitly recited, one or more additional embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A method of programming a therapeutic device, comprising the steps:
providing a programmable therapeutic device;
providing a therapy script authoring template device;
wherein said programmable therapeutic device comprises a central processing unit and is configured to provide one or more therapy techniques to a user;
creating on said therapy scripts authoring template device one or more therapeutic scripts;
receiving by said programmable therapeutic device at least one of said one or more therapeutic scripts; and
running on said programmable therapeutic device said at least one received therapeutic script.

2. The method of claim 1, wherein said one or more therapy techniques provided by said programmable therapeutic device are selected from the group of therapy techniques selected from one or more of: vibration; pulse; percussion; oscillation; electrical stimulation; and combinations thereof.

3. The method of claim 1, wherein said therapy script authoring template device comprises a therapy script authoring template software program;
wherein said therapy script authoring template software program comprises a graphical user interface;
wherein said graphical user interface displays a strength adjustment and a speed adjustment;
wherein said step of creating one or more therapeutic scripts comprises the steps of:
adjusting a strength; and
adjusting a speed.

4. The method of claim 1, wherein said step of receiving by said programmable therapeutic device of at least one of said one or more therapeutic scripts is via a wireless connection.

5. The method of claim 1, further comprising the steps:
providing one or more additional therapy script authoring template devices;
creating on said one or more additional therapy script authoring template devices a plurality of additional therapy scripts; and
storing in an online database said plurality of additional therapy scripts.

6. The method of claim 5, further comprising the step:
accessing by said user said plurality of additional therapy scripts.

7. The method of claim 6, wherein said step of accessing of said plurality of additional therapy scripts is via a subscription service.

8. The method of claim 5, wherein at least one of said plurality of additional therapy scripts is created by a massage therapy professional.

9. A programmable therapy system, comprising:
a programmable therapeutic device; and
a therapy script authoring template device;
wherein said programmable therapeutic device comprises a central processing unit and is configured to provide one or more therapy techniques to a user;
wherein said therapy scripts authoring template device creates one or more therapeutic scripts; and
wherein said at least one of said one or more therapeutic scripts are configured to be received and run on said programmable therapeutic device.

10. The system of claim 9, wherein said one or more therapy techniques provided by said programmable therapeutic device are selected from the group of therapy techniques selected from one or more of: vibration; pulse; percussion; oscillation; electrical stimulation; and combinations thereof.

11. The system of claim 9, wherein said therapy script authoring template device comprises a therapy script authoring template software program;
wherein said therapy script authoring template software program comprises a graphical user interface;
wherein said graphical user interface displays a strength adjustment and a speed adjustment;
wherein when said one or more therapeutic scripts are created a strength is adjusted and a speed is adjusted.

12. The system of claim 9, wherein said one or more therapeutic scripts received by said programmable therapeutic device is via a wireless connection.

13. The system of claim 9, further comprising:
one or more additional therapy script authoring template devices;
wherein said one or more additional therapy script authoring template devices create a plurality of additional therapy scripts; and
wherein said plurality of additional therapy scripts are stored in an online database.

14. The system of claim 13, wherein said user accesses said plurality of additional therapy scripts from said online database.

15. The system of claim 14, wherein said user accesses said plurality of additional therapy scripts is via a subscription service.

16. The system of claim 13, wherein at least one of said plurality of additional therapy scripts is created by a massage therapy professional.

17. The system of claim 16, wherein said massage therapy professional is also provided with a plurality of personal health information about said user.

18. The system of claim 11, wherein said programmable therapeutic device is a portable handheld massage device that comprises:
a massage head;
a memory; and
a motor.

19. The system of claim 18, wherein said graphical user interface further displays a length adjustment;
wherein when said one or more therapeutic scripts are created a length is adjusted.

20. The system of claim 19, wherein said graphical user interface further displays a plurality of attachments to select.
